(19) United States
(12) Patent Application Publication
(10) Pub. No.: US 2010/0057133 A1
(43) Pub. Date: Mar. 4, 2010

(54) Tibia-talus-calcaneus (T-T-C) locking plate

(76) Inventor: William H. Simon, Virginia Beach, VA (US)

Correspondence Address: GUERRY LEONARD GRUNE, 784 S VILLIER CT., VIRGINIA BEACH, VA, 23452, US

(21) Appl. No.: 12/229,653

(22) Filed: Aug. 26, 2008

(51) Int. Cl. A61B 17/70; A61B 17/04

(52) U.S. Cl. 606/280; 606/286; 606/301

(57) ABSTRACT

Disclosed is a bone fusion or implant, method and system for arthrodesis of a human ankle fusing the distal end of a tibia, talus and/or a calcaneus bone by employing a single anatomically lateral incision to the tibia, fibula, talus and calcaneus region, removing some or all of a fibula in the incision area and attaching the implant to the tibia, talus and/or calcaneus in a generally central area of the ankle. The removed portion of the fibula is morsilized and made into a paste and mixed with an autologous platelet concentrate that is applied as a bone graft to the implant site thereby enhancing the arthrodesis site and enhancing hemostasis.

### FIELD OF DISCLOSURE

The field of the present invention relates to a method and device for repairing or fusing damaged, deteriorating, or fractured tibia, talus and calcaneus bones in the ankle region. More particularly, the present invention relates to both a method and device for treating tibia, talus and/or calcaneus bones that are affected by illness or injury using lateral incision(s) and joint fusion(s).

### BACKGROUND OF DISCLOSURE

Implants are foreign devices which may be inserted into any bone. The utilization of implants in the treatment of bone fractures with plates, screws, intramedullary nails and rods are well known to those in the art. Various types of implants exist in the prior art, each composed of different materials and having different shapes with various degrees of functionality.

Intramedullary nails are adapted for insertion into the medullary canal of a bone or bones. Intramedullary nails have been limited in their application to long or large bones and such use has been widely known for long or large bones of the upper extremities (humerus, radius, ulna) and lower extremities (femur, tibia, fibula). Use of intramedullary nails allows physicians to secure fractured bones, maintain a desired length, and prohibit rotary motion while the bone heals and has time to rehabilitate. Intramedullary nails are also used for the fusion of bones. In an arthrodesis model the tibia, talus and calcaneus fracture repair failed at lower loads over time, resulting in cracks or breaks, as compared to blade. (Chiodo, Christopher P., Acevedo, Jorge I., Sammarco, V. James, Parks, Brent G., Boucher, Henry R., Myerson, Mark S., Schon, Lew C.; “Intramedullary Rod Fixation Compared with Blade-Plate-and-Screw Fixation for Tibiotalocalcaneal Arthrodesis: A Biomechanical Investigation”; The Journal of Bone & Joint Surgery, Vol. 85-A, Number 12, Pgs.2425-2428, December 2003)

Plates that incorporate a series of holes for screw insertion are also typical implants for the fixation of bones and joints. Plates to be used may be linearly straight or may have some curvatures or undulations, depending on the anatomical location and bone size. These curvatures or undulations are for placement for conforming to the bone circumferential shape and/or diameter. The hole positioning may provide for straight or angular insertion of the fasteners or screws that are to be used. Fasteners generically include all attaching means by which an implant may be attached to or connected with bone.

Such fasteners are inserted through a fastener hole or slot, also described as transfixation holes and screw holes. The use of plate locking screws is optional depending upon the severity of the bone damage. Static locking, by insertion of screws in proximal and distal locations, neutralizes rotational stresses while preventing shortening of the limb.

Generally, tibiotalocalcaneal fixation with plates requires either anterior and/or posterior incision and anterior and/or posterior placement of the respective implant device in relation to the T-T-C interface. One risk factor involving anterior implantation is that the implant is placed close to the skin and therefore there is increased risk of wound dehiscence, and ultimately, removal of the implant.

While there is much in the art regarding implants, there is a need for a lower infection risk implant and method for arthrodesis of the T-T-C region of a human foot. Such an implant and procedure may likely have any or all of the similar properties, features, and characteristics of the above mentioned implant methodologies, but greatly reduces the risk of wound dehiscence by providing an implant that is further from the skin surface than the present anterior implant methodologies.

### RELEVANT ART

U.S. Pat. No. 6,974,461, to Wolter, Dietmar, and unassigned, describes a fixation system for bones comprising: a force support having a posterior surface and an anterior surface; at least two holes in the force support each having an axis; and at least two bone screws each having a screw head portion and a non-head portion; wherein the bone screws are adapted to be inserted into the holes, where at least one hole has at least one deformable element which can become deformed by turning a bone screw into a hole, whereby the deformable element fixes a bone screw into a hole, where the deformable element is one selected from the group consisting of: a ridge, a lip or and an edge, and where the deformable element extends in a plane oriented obliquely to the axis of the hole.

U.S. Pat. No. 6,322,562, to Wolter, Dietmar, and unassigned, describes a fixation system for bones comprising: a connection carrier with at least one passage hole; at least one bone screw inserted into the at least one passage hole; the connection carrier including a seat surface and the bone screw including a seat surface, permitting a mutual alignment at various angles for fixing the bone screw at a certain angle to the connection carrier, the bone screw further including a preformed thread below the bone screw seat surface, the preformed thread deforming a portion of the passage hole below the seat surface of the connection carrier when the bone screw is screwed in so that a thread connection is formed between the bone screw thread and the connection carrier, the deformation being formed by rotating the bone screw at a certain angle to the connection carrier.

U.S. Pat. No. 4,794,918, to Wolter, Dietmar, and unassigned, describes a bone plate arrangement comprising:a bone plate including at least one bone screw hole having a center line and a seat on the upper side of the bone plate opposite the lower side adapted to face the bone, said seat having an acutely narrowing profile in the direction from the upper toward the lower side; a bone screw having a longitudinal axis, said bone screw adapted to pass through the bone screw hole into the bone and including a bone screw head having a generally convex seating surface which is contoured such that when the bone screw is fully inserted into the bone through the hole with the axis along a selected one of a range of angular orientations relative to said center line, the seating surface of the head is supported in said seat; a cover plate including means for engaging the bone plate so that the underside of the cover plate tightly engages the screw head; and said bone screw head and the underside of the cover plate having raises and recesses which mutually engage when the cover plate tightly engages the screw head so as to hold the head in fixed lateral relation to the bone plate and prevent pivoting thereof, whereby the bone screw axis is fixed in said selected angular orientation relative to the hole centerline.

U.S. Pat. No. 4,705,031, to Wolter, Dietmar, and unassigned, describes an osteosynthesis plate for the pressure stabilization of bone fragments, comprising; a base plate for spanning the bone fragments, the base plate having a first end adapted to lie on a first bone fragment and an opposed second end adapted to be secured on a second bone fragment; at least one slot in the first end of the base plate, each slot being elongated in the direction between said plate ends, each slot having a plurality of screw hole sections adapted to receive a bone screw, the sections lying end-to-end on a uniform pitching spacing; each of said screw hole sections including a ramp sloped downwardly in the direction toward the second end of the base plate, the ramp having a useful ramp length defined by the distance the base plate would move relative to the first bone fragment as a bone screw is advanced into the bone fragment from an initial position at the top of the ramp to a final position at the bottom of the ramp; wherein the pitch for the screw hole sections in a given slot is no greater than the number of slots in said first end of the plate, multiplied by the useful ramp length.

U.S. Patent Publication No. US20070055234A1, to McGrath, et. al., and unassigned, describes a method of reconfiguring an external fixator that is stabilizing bone, comprising: selecting a subject having one or more bones fixed by an external fixator including a frame having a pair of frame members connected to the one or more bones and a plurality of struts defining a substantially fixed relative position of the pair of frame members; bracing the frame with a brace; reconfiguring the frame while the brace restricts movement of the pair of frame members; and removing the brace after the step of reconfiguring such that the external fixator with the reconfigured frame fixes the one or bones without the brace.

PCT Patent Publication No. WO07035713A2 to Kaelblein, et. al., and assigned to Small Bone Innovations, Inc., describes a condylar plate for fixation of a small bone fracture comprising two or more tines and a plate integral with tines and comprising one or more apertures configured to receive a fastener. The tines of the condylar plate are configured to be fixed to the condyle portion of the small bone. Additionally is a method of fitting a condylar plate to a small bone in a patient, with the method comprising molding a pliable condylar plate template having a shape that corresponds to a shape of the condylar plate to the small bone. The template comprises a longitudinal planar shaft substantially identical to a longitudinal planar shaft of the condylar plate and the template comprises apertures in a position substantially identical to a location of the tines are located in the condylar plate. Drilling holes through the apertures into the bone to indicate where the tines of the condylar plate will be fixated to the bone and bending the condylar plate to conform to the template configuration.

PCT Patent Publication No. WO06007965A1 to Wolter, Dietmar, and unassigned, describes an invention relating to a bone-fixation system comprising a connecting support that contains several bores and at least one bone screw that can be inserted into a bore. The system is characterised in that it comprises at least one filler element that can be inserted into a bore, in addition to means for securing the filler element in the bore.

Russian Patent Publication No. RU2226079C2, to Konjukhov, et. al., and unassigned, describes an method involves performing arthrodesis of talonavicular, calcaneocuboid and talocalcanean joints, separating from attachment place together with cortical plate of short finger extensors, filling with bone autotransplants produced when making articulation resection of free interosseous spaces, stretching anterior tibial muscle tendons, common finger extensor and short tibial muscle tendons, shortening Achilles tendon and transplanting long tibial muscle tendon on calcanean tuber, setting dislocation in the first metatarsophalangeal joint in open way. The achieved disposition is fixed by means of wires conducted through calcaneus, talus and distal epiphysis of tibia, through talus, navicular bone, calcaneus and cuboid bone and the first finger and the first metatarsal bone.

Russian Patent Publication No. RU2193366C2, to Dolgopolov, et. al., and assigned to Rossijskij Nauchno-Isseldovatel'skij Institut Trav, describes a method involves applying Ilizarov apparatus over the talocrural articulation area at the first stage. Distraction is carried out until the lost distance between the calcaneus and the tibia is recreated. Fibula osteotomy is carried out at the second stage without taking off the Ilizarov apparatus and necrotized areas of the talus are removed. So a created defect is substituted with an iliac bone autograft and fibula osteosynthesis is carried out.

### SUMMARY OF THE DISCLOSURE

An object of the present disclosure is to provide a bone fusion or implant device, method and system for inserting an implant in a human ankle, fusing the distal end of a tibia, the talus and/or calcaneus bone by employing an anatomically lateral incision to the tibia, talus, fibula and calcaneus region, removing some or all of a fibula in the incision area, attaching the implant to the tibia, talus and/or calcaneus in a generally central area of the ankle allowing for fusing the bones together, making a cortical-calcaneus bone graft for any of the bones where such a graft is useful including a portion of the fibula, applying the cortical-calcaneus bone graft as a packing of the anterior and posterior regions to achieve an extra-articular arthrodesis to assist in fusion. The implant is generally centrally located within the confines of an ankle region for stabilizing the T-T-C. These devices and procedure increases the ability to achieve successful and sustainable arthrodesis.

Another objective is to attach the implant to the tibia, talus and/or calcaneus by means of rods, screws, pins, nails or other means of attachment.

Another objective of the disclosure is to remove some, or all of the fibula wherein a portion of the fibula that is removed may be morsilized into a paste, powder or slurry as a cortical-calcaneus graft mixed with an autologous platelet concentrate and packed over the implant as a means prevent wound dehiscence of the arthrodesis site. The platelets release growth factors to stimulate bone growth and help reduce hemorraging. Hemostasis is enhanced with platelet concentration.

An additional objective is to include an implant device that is malleable for forming around the contours of the tibia, talus and/or calcaneus and may be of a length and width to accommodate bone size and deformity and may be comprised of stainless steel, titanium or another biocompatible material.

Additionally the implant is initially formed of a biocompatible material wherein holes are placed at various locations along the implant and at various angles to the longitudinal and latitudinal plane of the implant enabling screws, rods, pins, nails or other devices securing the implant to the tibia, talus and/or calcaneus in various configurations including overlapping, parallel, divergent or convergent within a bone.

Another objective is to provide placement of the implant generally centrally within and interiorly separate from the skin to reduce the risk prominence of device (implant) and to prevent wound dehiscence.

Another objective of the disclosure is to provide an implant whereby the distal end of the implant bends at a thirty-degree (30°) angle or within a fifteen (15°) to forty-five (45°) degree angle in a posterior direction for attachment to the calcaneus.

Another objective is to provide an implant device in the form of a locking plate and screw fixation device to the tibia, talus and/or calcaneus.

Another objective is to provide an implant for the arthrodesis of severe ankle and subtalar arthrosis, talar collapse, avascular necrosis, hindfoot deformity, stage 4 flatfoot and/or Charcot deformity.

Another objective is to provide an anatomically laterial incision site whereby an anteriolateral and/or posteriolaterial implant or grafting may be inserted directly laterally for the stabilization of the tibia, talus and/or calcaneus.

### BRIEF DESCRIPTION OF THE DRAWINGS

FIG. 1A is an isometric drawing of the T-T-C implant.

FIG. 1B is an isometric view of a fastener in this case a typical orthopedic screw.

FIG. 2A is a lateral skeletal view of the foot showing the tibia, fibula, talus and calcaneus as well as the midfoot region and phalanges.

FIG. 3 is a lateral skeletal view of the foot showing the tibia, fibula, talus and calcaneus showing the implant fusing the tibia, talus and calcaneus.

FIG. 4 is an anterior view of the tibia, talus and calcaneus showing the implant and phantom views of the fasteners within the T-T-C region.

### DETAILED DESCRIPTION OF THE DRAWINGS

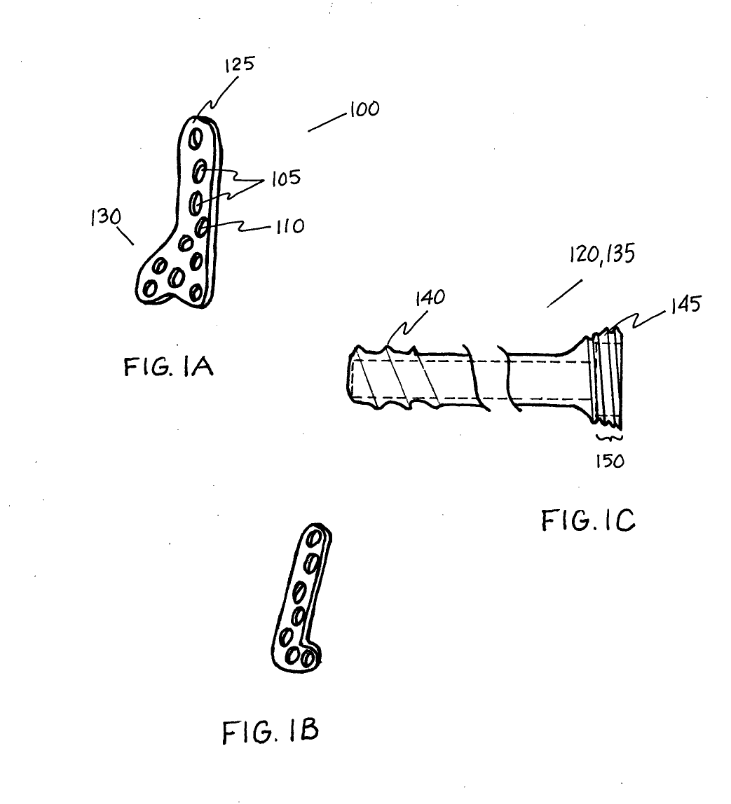

FIG. 1A is an isometric view of a T-T-C implant [100] showing the holes [105] which may have internal threads [110] for a locking feature [115] that would lock a fastener [120] (Ref. FIG. 1C) to the implant [100]. The holes [105] may be perpendicular to the longitudinal surface [125] of the implant [100] or may be angled to provide a fastener direction of various configurations that are overlapping, parallel, divergent or convergent.

The implant [100] also has a distal end [130] that is larger than the longitudinal plane [125] that is angled at, or about, thirty degrees within a range of fifteen to forty five degrees. The distal end [130] also contains holes [105] of various configurations that provide direction for a fastener [120] that are overlapping, parallel, divergent or convergent.

The implant [100] is made of a biocompatible material that would typically be titanium or stainless steel and be malleable to be shaped to meet the contours and undulations of a patients bone structures and shapes.

FIG. 1B is an isometric view of a T-T implant [155] which has a distal end that is angled at ninety-degrees to the longitudinal plane [125].

FIG. 1C is an isometric view of a fastener [120], in this case a typical orthopedic screw [135]. The screw [135] has a wide spiral thread [140] for insertion into a bone and a narrow external thread [145] at the head [150] that secures with the hole internal threads [110] (Ref. FIG. 1A) of the implant [100] (Ref. FIG. 1A).

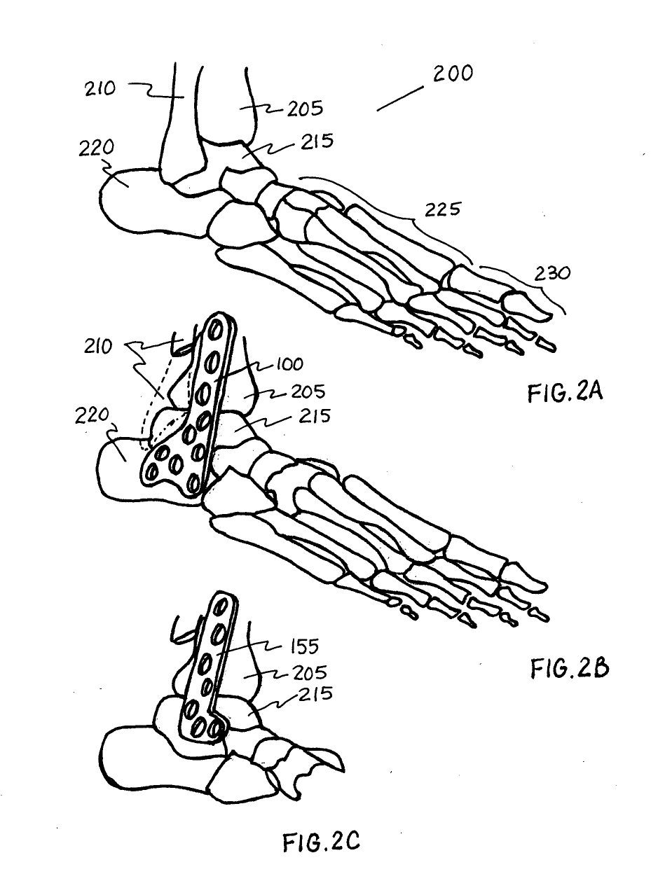

FIG. 2A is a lateral skeletal view of the right foot [200] showing the distal end of the tibia [205], distal end of the fibula [210], talus [215] and calcaneus [220] as well as the midfoot region [225] and phalanges [230].

FIG. 2B is a lateral skeletal view of the right foot [200] showing the implant [100] fastened to the tibia [205], talus [215] and calcaneus [220] using screws [135] to secure the implant [100]. A portion of the fibula [210] is shown removed allowing for access to the lateral aspect of the tibia [205] as well as the implant [100] being centrally located within the distal portion of the leg away from the skin (not shown).

The portion of the fibula [210] which has been removed is ground or pulverized to form a cortical-calcaneus bone graft packing [240] (not shown) that may be mixed with an autologous platelet concentrate and packed over the fastened implant [100] to aid in hemostasis. The lateral fastening of the implant [100] acts to secure, or fuse, the tibia [205], talus [215] and calcaneus [220] and act as an implant and stabilize the fusion of the skeletal structure.

FIG. 2C is a lateral skeletal view of the right foot [200] showing the implant T-T [155] fastened to the tibia [205] and talus [215] only.

An alternative method would be to reshape the implant [100] to secure the tibia [205] and calcaneus [220] should the talus [215] be removed.

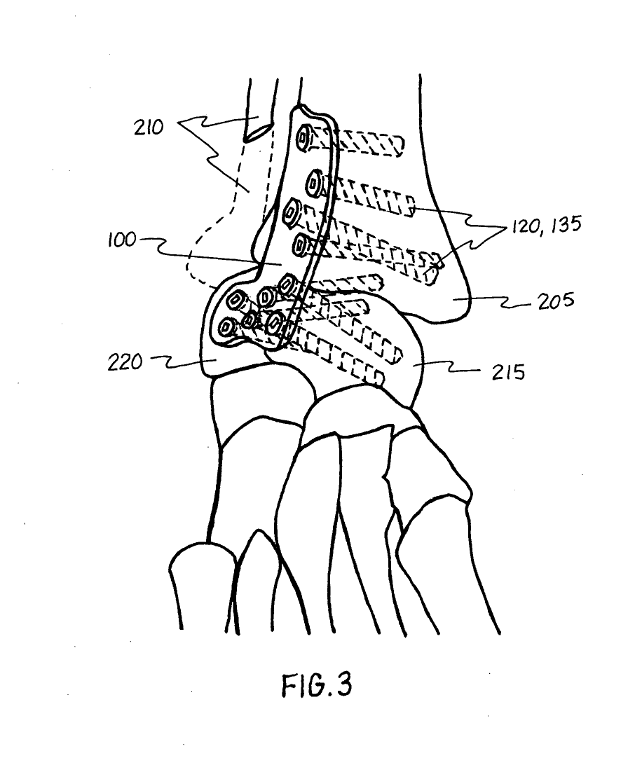

FIG. 3 is an anterior view of the tibia [205], talus [215] and calcaneus [220] showing the implant [100] and phantom views of potential locations of the fasteners [120] or screws [135] within the T-T-C region. The fibula [210] is partially removed.

### Overview of Surgical Technique for T-T-C (Tibial-Talar-Calcaneal), T-T (Tibial-Talar) and T-C (Tibial-Calcaneal) Arthrodesis

In the present disclosure the patient is placed on the operating room table in the supine (face up) position. As prior art, the posterior approach to T-T-C arthrodesis requires a prone position (face down) which is more dangerous for the patient from an anesthesia perspective. It is also more difficult to align the patient's lower extremity in the correct position with the patient face down.

An extensive anatomically lateral incision is performed over the fibula exposing the distal twelve (12) to fourteen (14) centimeters of the fibula. This section of fibula is surgically removed and cleaned of all soft tissue. The fibula section is then morsilized and mixed with autologous platelet concentrate to form a bone graft for additional healing in the arthrodesis site. The posterior approach requires a second surgical site for harvesting bone graft frequently from the hip.

The T-T-C, T-T or T-C joint surfaces are debrided of all articular cartilage so raw bone is exposed for the arthrodesis. If the talus exhibits avascular necrosis or is destroyed, a tibial-calcaneal arthrodesis can be performed either without an interpositional graft or with an interpositional graft such as a femoral head in order to maintain length of the extremity.

The foot is placed in alignment with the second metatarsal aligned with the tibial crest and the ankle at zero degrees of dorsiflexon. A temporary Steinman pin is placed from the plantar aspect of the heel into the distal tibia. X-rays are taken at this time to check alignment.

Cannulated screws are placed from the distal tibial metaphysis across the arthrodesis sites to get compression. One cannulated screw is placed from the distal anterior tibia into the calcaneus posteriorly. The second screw is placed from the posterior tibia into the medial column of the foot. Placement of these screws are performed under fluoroscopic guidance and through the single lateral incision site.

The lateral T-T-C, T-T or T-C locking plate implant is contoured to the patient's anatomy. A large fixed angle locking screw is placed first into the sustentaculum of the calcaneus which is the densest part of the bone. Polyaxial screws are then placed for additional fixation into the calcaneus. For additional compression a non-locking screw can be placed by eccentric drilling or a compression device can be applied to the tibia and plate. The remaining fixed angle locking screws are inserted and secured. Fluoroscopic evaluation is performed to check screw lengths and alignment.

The bone graft that was harvested from the fibula and processed is used to fill in any deficits and placed posteriorly through the lateral incision for an extra-articular arthrodesis. The tissues are closed in layers and a drain is placed to prevent post-operative hematoma. Removal the fibula ensures there is adequate soft tissue coverage of the locking plate. An anterior approach with a locking plate manufactured by Synthes has very little soft tissue coverage which can lead to wound dehiscence or pain from the hardware. A post-operative dressing is placed along with a posterior splint. The drain is left in for 24 to 48 hours. Dressings are changed and the patient is placed into a non-weight bearing fracture brace until healing has occurred on X-ray. Healing time is approximately six to twelve weeks.

What is claimed is:

1. An implant for inserting into a human ankle for arthrodesis of the distal end of a tibia, a talus and/or a calcaneus comprising;
a locking-plate and screw fixation device with a thirty degree bend posteriorly on a distal end of said implant wherein the angle is in the range of fifteen to forty five degrees.

2. The implant of claim 1, wherein said implant is biocompatible material comprising titanium, stainless steel or other said biocompatible material and formable to the contour, shape and size of said tibia, said talus and said calcaneus.

3. The implant of claim 1, wherein said implant comprises holes at various locations along said implant and said holes are provided at various angles to said longitudinal surface and said distal end and wherein said holes optionally include interior threads.

4. The implant of claim 3, wherein said holes at various angles guide said fasteners for specific location within said tibia, said talus and/or said calcaneus such that said fasteners are overlapping, parallel, divergent or convergent and said fasteners optionally comprise exterior threads that mate with said interior threads of said holes to secure said fasteners to said implant.

5. The implant of claim 1, wherein said lateral incision to said ankle provides ease of access to said ankle region of said tibia, fibula, said talus and said calcaneus.

6. The implant of claim 1, wherein a portion of said fibula is removed and morsilized or pulverized to form a paste, powder or slurry that may be mixed with an autologous platelet concentrate to form a bone graft.

7. The implant of claim 6, wherein a portion of said removed fibula from the distal end is twelve to fourteen centimeters in length.

8. The implant of claim 1, wherein said fastener is a rod, screw, pin, and/or nail.

9. The implant of claim 1, wherein said implant is in a generally central location and is laterally placed where there is adequate soft tissue coverage.

10. The implant of claim 1, wherein said implant is utilized for the arthrodesis of severe ankle and subtalar arthrosis, talar collapse, avascular necrosis, hindfoot deformity, stage 4 flatfoot and/or Charcot deformity.

11. The implant of claim 1, wherein said lateral incision to said ankle provides ease of access to said ankle region whereby said implant and/or said bone graft may be inserted directly laterally into an anteriolateral and/or posteriolaterial position.

12. An implant for inserting into a human ankle for arthrodesis of the distal end of a tibia, a talus and/or a calcaneus comprising;
employing a single anatomically lateral incision to an ankle region of said tibia, fibula, said talus and said calcaneus,
removing some or all of said fibula in said ankle region,
attaching said implant with fasteners to said tibia, said talus and/or said calcaneus in a generally central location within said ankle region thereby fusing said tibia, said calcaneus and/or said talus together stabilizing said ankle,
making a paste derived of any or all of removed said fibula,
applying said paste of said fibula as a bone graft to said implant, said tibia, said talus and/or said calcaneus, thereby enhancing the ability to achieve arthrodesis and hemostasis.

13. A method for inserting into a human ankle for arthrodesis of the distal end of a tibia, a talus and/or a calcaneus comprising;
placing a patient on the operating room table in the supine position,
performing an extensive anatomically lateral incision over the fibula exposing the distal end twelve (12) to fourteen (14) centimeters of the fibula
surgically removing and cleaning all soft tissue,
morsilizing and mixing with autologous platelet concentrates thereby forming bone graft providing additional healing in the arthrodesis site,
debriding the T-T-C, T-T or T-C joint surfaces of all articular cartilage so raw bone is exposed for the arthrodesis such that if the talus exhibits avascular necrosis or is destroyed, a tibial-calcaneal arthrodesis can be performed either without an interpositional graft or with an interpositional graft such as a with a femoral head in order to maintain length of the extremity,
placing the foot in alignment with the second metatarsal aligned with the tibial crest and the ankle at zero degrees of dorsiflexon,
providing a temporary Steinman pin placed from the plantar aspect of the heel into the distal tibia,
optionally taking X-rays at this time to-check alignment,
placing cannulated screws from the distal tibial metaphysis across the arthrodesis sites to obtain compression, wherein placing at least one cannulated screw from the distal anterior tibia into the calcaneus posteriorly is accomplished and wherein placing a second screw from the posterior tibia into the medial column of the foot is performed under fluoroscopic guidance and through the single lateral incision site.

14. The method of claim 13, wherein the lateral T-T-C, T-T or T-C locking plate implant is contoured to the patient's anatomy and wherein a large fixed angle locking screw is placed first into the sustentaculum of the calcaneus

15. The method of claim 13, followed by placing polyaxial-screws for additional fixation into the calcaneus and for additional compression placing a non-locking screw by eccentric drilling or applying a compression device to the tibia and applying a plate allowing the remaining fixed angle locking screws to be inserted and secured, followed by evaluating and checking screw lengths and alignment by fluoroscopy.

16. The method of claim 13, wherein using the bone graft harvested from the fibula and processed to fill in any deficits and placed posteriorly through the laterial incision for an extra-articular arthrodesis is performed and wherein tissues are closed in layers and a drain is properly placed to prevent post operative hematoma.

17. The method of claim 13, wherein performing removal of the fibula is performed ensuring there is sufficient soft tissue coverage of the locking plate.

18. The method of claim 13, wherein placing a post-operative dressing is accomplished along with a posterior splint and wherein the drain is left in for 24 to 48 hours.

19. The method of claims 13-18, wherein once the operation is complete, dressings are changed and the patient is placed into a non-weight bearing fracture brace until healing has occurred based upon X-ray analysis and wherein standard healing time is approximately six to twelve weeks.
